(19) United States
(12) Patent Application Publication
(10) Pub. No.: US 2015/0148902 A1
(43) Pub. Date: May 28, 2015

(54) Nasal Implant

(71) Applicant: Sampandh KOMRIT, (US)

(72) Inventor: Sampandh Komrit, Samut Prakan (TH)

(21) Appl. No.: 14/405,130

(22) PCT Filed: Aug. 23, 2013
(86) PCT No.: PCT/TH2013/000039

§ 371 Date: Dec. 2, 2014

Related U.S. Application Data
(60) Provisional application No. 61/771,517, filed on Mar. 1, 2013.

(51) Int. Cl. A61F 2/18

(52) U.S. Cl. CPC A61F 2/186; A61F 2220/0008; A61F 2240/002
USPC 623/10; 83/13

(57) ABSTRACT

Present example embodiments relate generally to nasal implants comprising a body portion, the body portion forming a central axis and having opposing inner body and outer body surfaces. The inner body surface may comprise a ventral groove portion disposed substantially parallel to the central axis, and the outer body surface may comprise a ridge portion. The ventral groove portion may comprise a deepest portion in proximate correspondence to a highest portion of the ridge portion with respect to the central axis. The nasal implant may comprise an elongated tail portion. A lower portion of the tail portion may be in communication with at least an upper portion of the body portion. An angle formable between an axis definable by the tail portion and the central axis may be between 0 to 90 degrees. The nasal implant may comprise a first wing portion, head portion, second wing portion, and leg portion.

### TECHNICAL FIELD

The present disclosure relates generally to nasal implants, including those for use in rhinoplasty.

### BACKGROUND

Rhinoplasty is a procedure pertaining to nasal reconstruction performed as a result of one or more of a variety of reasons including, but not limited to, injury, congenital defects, and personal aesthetic desire. Rhinoplasty includes nasal augmentation, and such augmentation often involves implantation of a nasal implant.

To date, technological advances and improvements in nasal implants have been generally directed to material or composition improvements. However, little progress has been made relating to improvements to the functional and aesthetic portions, features, and designs of nasal implants.

Presently, nasal implants come in a limited variety of shapes and sizes, including cursorily hand-crafted rectangular prism shaped nasal implants, which are very common among those of lay surgeons, and symmetrically shielded, L-shaped nasal implants.

It is recognized herein that patients, that is, recipients of nasal implants, more often than not encounter one or more undesirable complications with their implanted nasal implants, including visible depressions and/or lines in or about the edges of the implanted nasal implant; concave-shaped nasal dorsum; undesirably wide dorsum due to, among other things, a lack of understanding and consideration by the surgeon and/or manufacturer/designer of the nasal implant of the variations in thickness of relevant areas of the nose; displacement of the nasal implant after implantation, including vertical, horizontal, and/or rotational displacement; inflexibility of one or more portions of the implanted nasal implant; and more serious consequences, including causing perforations to one or more parts of the nose, including the tip of the nose, and causing permanent deformity resulting from sinking and/or breaking into the gap between parts of the nose, including those between the lower lateral cartilages.

### SUMMARY

Present example embodiments relate generally to a nasal implant comprising an elongated body portion, an elongated tail portion, a first wing portion, a head portion, a second wing portion, and a leg portion. The body portion forms a central axis and comprises opposing inner body and outer body surfaces, the inner body surface having a ventral groove portion disposed substantially parallel to the central axis, and the outer body surface having a ridge portion. The ventral groove portion comprises a deepest portion in proximate correspondence to a highest portion of the ridge portion with respect to the central axis. A lower portion of the tail portion is in communication with at least an upper portion of the body portion, wherein an angle formable between an axis definable by the tail portion and the central axis is between 0-90 degrees. The first wing portion comprises opposing inner first wing and outer first wing surfaces, wherein an inner portion of the first wing portion is in communication with at least a first outer portion of the body portion. The head portion comprises opposing inner head and outer head surfaces, wherein the inner head surface is in communication with at least a lower portion of the inner body surface and the outer head surface is in communication with at least a lower portion of the outer body surface. The second wing portion comprises opposing inner second wing and outer second wing surfaces, wherein an inner portion of the second wing portion is in communication with at least a first outer portion of the head portion. The leg portion comprises opposing inner leg and outer leg surfaces, an upper portion of the leg portion in communication with at least a lower portion of the head portion.

In another example embodiment, a nasal implant comprises an elongated body portion and an elongated tail portion. The body portion forms a central axis and comprises opposing inner body and outer body surfaces, the inner body surface having a ventral groove portion disposed substantially parallel to the central axis, and the outer body surface having a ridge portion. The ventral groove portion comprises a deepest portion in proximate correspondence to a highest portion of the ridge portion with respect to the central axis. A lower portion of the tail portion is in communication with at least an upper portion of the body portion, wherein an angle formable between an axis definable by the tail portion and the central axis is between 0-90 degrees.

In another example embodiment, a nasal implant comprises an elongated body portion and a first wing portion. The body portion forms a central axis and comprises opposing inner body and outer body surfaces, the inner body surface having a ventral groove portion disposed substantially parallel to the central axis, and the outer body surface having a ridge portion. The ventral groove portion comprises a deepest portion in proximate correspondence to a highest portion of the ridge portion with respect to the central axis. The first wing portion comprises opposing inner first wing and outer first wing surfaces, wherein an inner portion of the first wing portion is in communication with at least a first outer portion of the body portion.

In another example embodiment, a nasal implant comprises an elongated body portion and a head portion. The body portion forms a central axis and having opposing inner body and outer body surfaces, the inner body surface having a ventral groove portion disposed substantially parallel to the central axis, and the outer body surface having a ridge portion. The ventral groove portion comprises a deepest portion in proximate correspondence to a highest portion of the ridge portion with respect to the central axis. The head portion comprises opposing inner head and outer head surfaces, wherein the inner head surface is in communication with at least a lower portion of the inner body surface and the outer head surface is in communication with at least a lower portion of the outer body surface.

In another example embodiment, a method is described for adapting a nasal implant for nasal augmentation. The nasal implant comprises an elongated body portion, an elongated tail portion in communication with at least an upper portion of the body portion, a first wing portion in communication with at least a first outer portion of the body portion, a head portion in communication with at least a lower portion of the body portion, a second wing portion in communication with at least an outer portion of the head portion, and a leg portion in communication with at least a lower portion of the head portion. The body portion forms a central axis and comprises opposing inner body and outer body surfaces, the inner body surface having a ventral groove portion disposed substantially parallel to the central axis, the outer body surface having a ridge portion, and the ventral groove portion comprising a deepest portion in proximate correspondence to a highest portion of the ridge portion with respect to the central axis. The method comprises identifying an appropriate set of dimensions of the nasal implant for the nasal augmentation, and adapting the nasal implant for the nasal augmentation by performing a trimming of at least an outer peripheral portion of at least one of the body portion, the tail portion, the first wing portion, the neck portion, the second wing portion, the head portion, and the leg portion.

In another example embodiment, a method is described for adapting a nasal implant for nasal augmentation. The nasal implant comprises an elongated body portion, an elongated tail portion in communication with at least an upper portion of the body portion, a first wing portion in communication with at least a first outer portion of the body portion, a head portion in communication with at least a lower portion of the body portion, a second wing portion in communication with at least an outer portion of the head portion, and a leg portion in communication with at least a lower portion of the head portion. The body portion forms a central axis and comprises opposing inner body and outer body surfaces, the inner body surface having a ventral groove portion disposed substantially parallel to the central axis, the outer body surface having a ridge portion, and the ventral groove portion comprising a deepest portion in proximate correspondence to a highest portion of the ridge portion with respect to the central axis. The method comprises identifying an appropriate set of dimensions of the nasal implant for the nasal augmentation, and adapting the nasal implant for the nasal augmentation by detaching at least one of the body portion, the tail portion, the first wing portion, the neck portion, the second wing portion, the head portion, and the leg portion.

### BRIEF DESCRIPTION OF THE DRAWINGS

For a more complete understanding of the present disclosure, example embodiments, and their advantages, reference is now made to the following description taken in conjunction with the accompanying drawings, in which like reference numbers indicate like features, and:

FIGS. 1 to 4 illustrate example isometric views of example embodiments of a nasal implant;

FIG. 5 illustrates an example rear view of example embodiments of a nasal implant;

FIG. 6 illustrates an example front view of example embodiments of a nasal implant;

FIG. 7 illustrates a first side view of example embodiments of a nasal implant;

FIG. 8 illustrates a top view of example embodiments of a nasal implant;

FIG. 9 illustrates a bottom view of example embodiments of a nasal implant;

FIG. 10 illustrates a cross-sectional view of example embodiments of a nasal implant;

FIGS. 11 to 16 illustrate cross-sectional views of example embodiments of a nasal implant;

FIG. 17 illustrates an example embodiment of modifying an example embodiment of a tail of an example embodiment of a nasal implant;

FIG. 18 illustrates an example embodiment of modifying a tail and wing of an example embodiment of a nasal implant;

FIG. 19 illustrates an example embodiment of modifying a neck of an example embodiment of a nasal implant;

FIG. 20 illustrates an example embodiment of modifying a neck and a head of an example embodiment of a nasal implant; and

FIGS. 21 and 22 illustrate example portions of an example embodiment of a nasal implant after modifying by detaching thereof.

Although similar reference numbers may be used to refer to similar elements for convenience, it can be appreciated that each of the various example embodiments may be considered to be distinct variations.

### DETAILED DESCRIPTION

Example embodiments will now be described hereinafter with reference to the accompanying drawings, which form a part hereof, and which illustrate example embodiments which may be practiced. As used in the disclosures and the appended claims, terms such as “example embodiment” and “present embodiment” do not necessarily refer to a single embodiment, although they may, and various example embodiments may be readily combined and interchanged, without departing from the scope or spirit of example embodiments. Furthermore, the terminology as used herein is for the purpose of describing example embodiments only and is not intended to be limitations. In this respect, as used herein, the term “in” may include “in” and “on”, and the terms “a”, “an” and “the” may include singular and plural references. Furthermore, as used herein, the term “by” may also mean “from”, depending on the context. Furthermore, as used herein, the term “if” may also mean “when” or “upon”, depending on the context. Furthermore, as used herein, the words “and/or” may refer to and encompass any and all possible combinations of one or more of the associated listed items.

Presently, nasal implants may be broadly categorized into three types. A first type of nasal implant comprises a short, substantially straight tail. As used herein, a tail will refer to the top part of a nasal implant. Typically, the angle of the tail relative to the remaining part of the nasal implant will be in or about 180 degrees, which, when implanted, will cause an unnatural look and feel in most patients.

A second type of nasal implant comprises protruding sides. Typical protruding sides comprise incorrect proportions and shapes, such as being overly thick and relatively hard to bend in a natural manner, and often causes inclining toward one or more sides of the nose when the nasal implant is implanted. It is recognized herein that typical protruding sides will cause obtrusive depressions when the nasal implant is implanted, especially in patients having relatively thin nasal skin.

A third type of nasal implant is of the type that is shielded and comprises an “L” shaped body. A shield is generally attached at the head of nasal implant, which corresponds to the portion of the nasal implant that presses against an inner surface of the tip of a patients' nose. However, it is recognized herein that even with the support of a shield, patients often suffer eventual perforation at the tip of the patients' nose because shields of such nasal implants typically have incorrect angles and/or sizes. For example, on the one hand, if the shield is not sufficiently large, the pressure at the tip of the patients' nose will be high and will cause said perforations over time. On the other hand, if the shield is overly large, the result will be aesthetically unpleasant and the overall weight of the nasal implant will be large, causing said perforations over time and/or other complications. In addition, if the shield is not fixedly implanted at a proper angle, then only a small portion of the shield will provide surface contact with the inner surface of the patients' nose, resulting in unduly high pressure at the said small portion of the inner surface of the patients' nose and, over time, causing said undesirable perforations.

Present example embodiments provide for one or more advantages over conventional nasal implants and implantation procedures thereof, including advantages over those problems described above and herein, and include, but are not limited to: preserving natural curvatures and/or angles in or about the root of a patients' nose; providing straight and naturally narrow nasal dorsa; substantially eliminating conspicuous depressions; providing for more secureness from displacements when the nasal implant is implanted; providing more natural flexibility and feeling when the nasal implant is implanted; substantially eliminating sinking and/or breaking into gaps between greater alar cartilages; substantially eliminating perforation in or about the tip of the nose; and enabling modifications and/or customizations based on the patients' needs and/or desires.

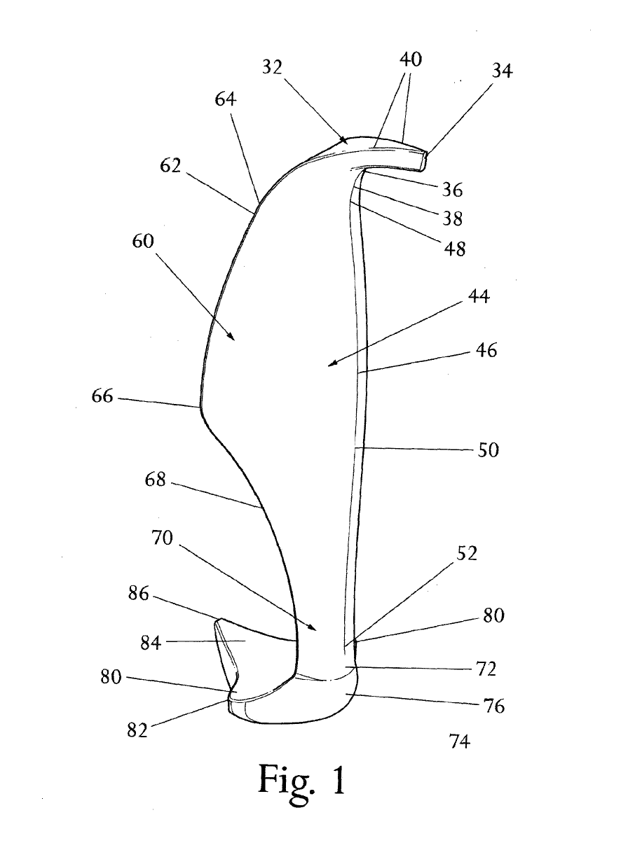

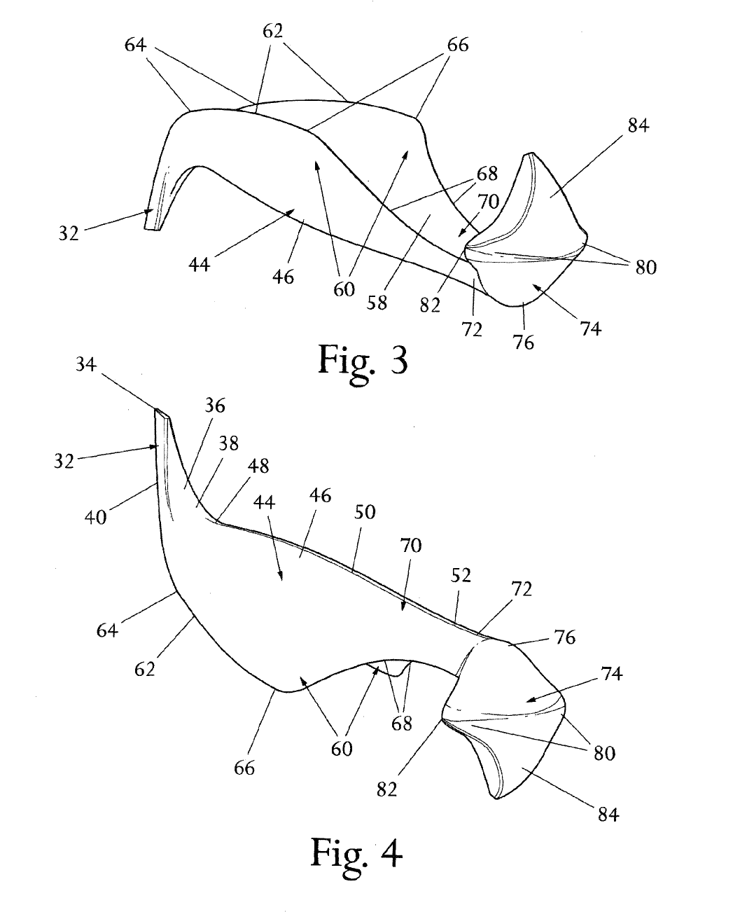

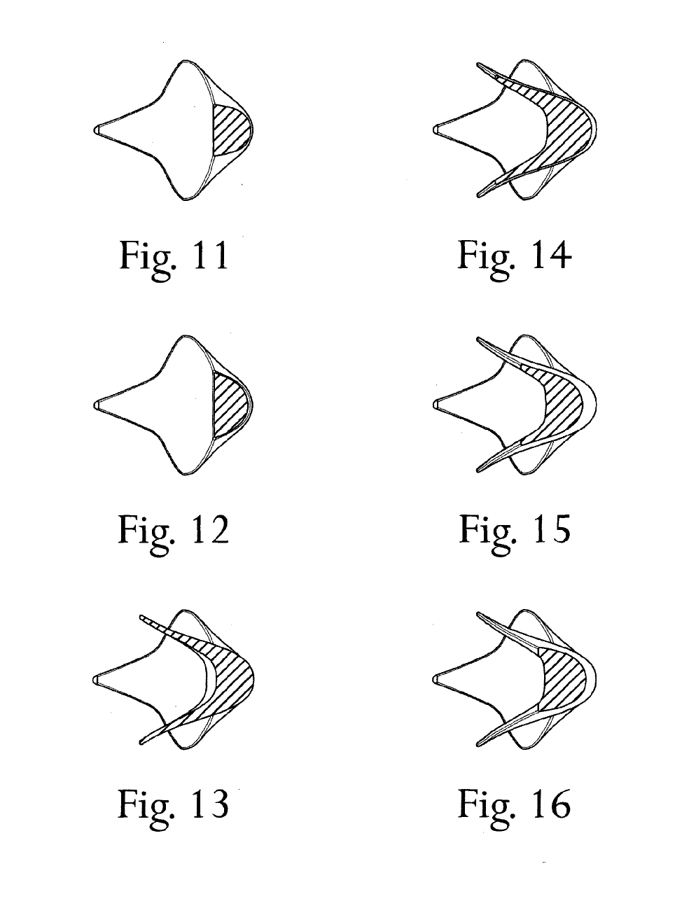

FIGS. 1-16 illustrate various perspective views of example embodiments of a nasal implant 100. Nasal implant 100 may be any one or more of a unitary body, a body comprising a plurality of fixedly secured and/or adjustable portions, an elongated body, and/or a body comprising one or more shapes and sizes, so long as the nasal implant provides for, among other things, structural support, naturally aesthetic appeal, and cooperation with at least one or more parts of the nose to achieve the advantages recited herein, including restricting and/or eliminating of undesirable translational movements, breaking, sliding, and/or coming apart of the nasal implant 100 from the patients' nose (not shown) when the nasal implant 100 is implanted.

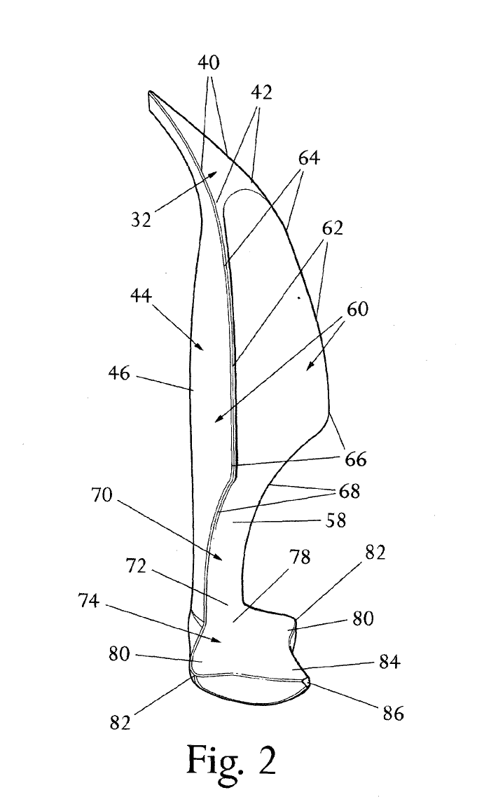

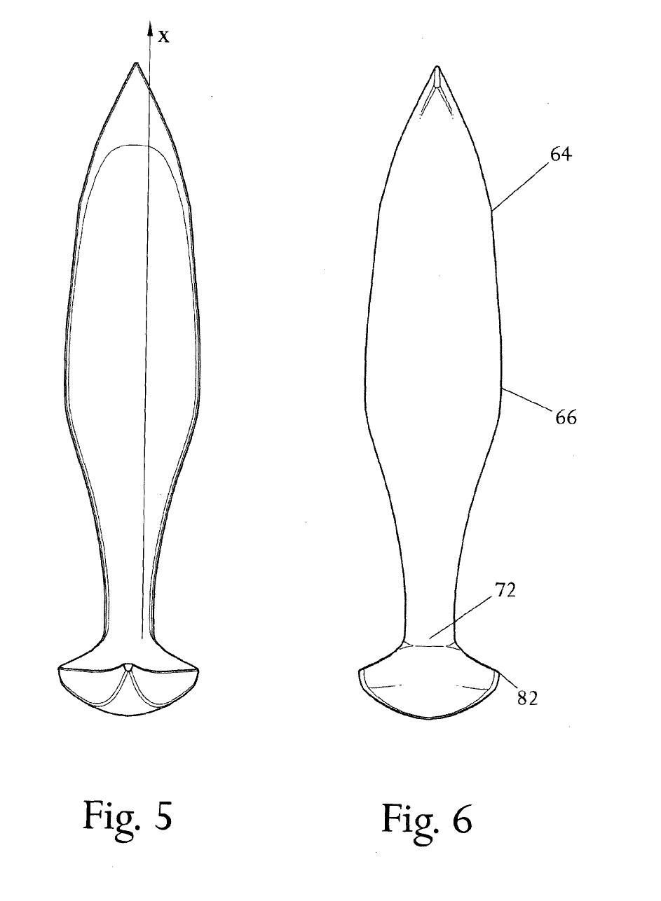

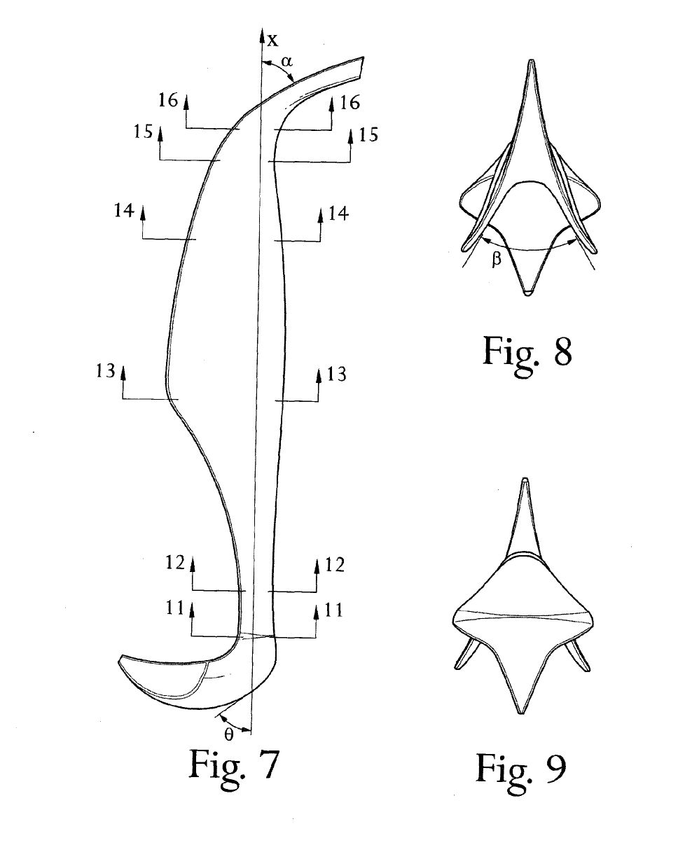

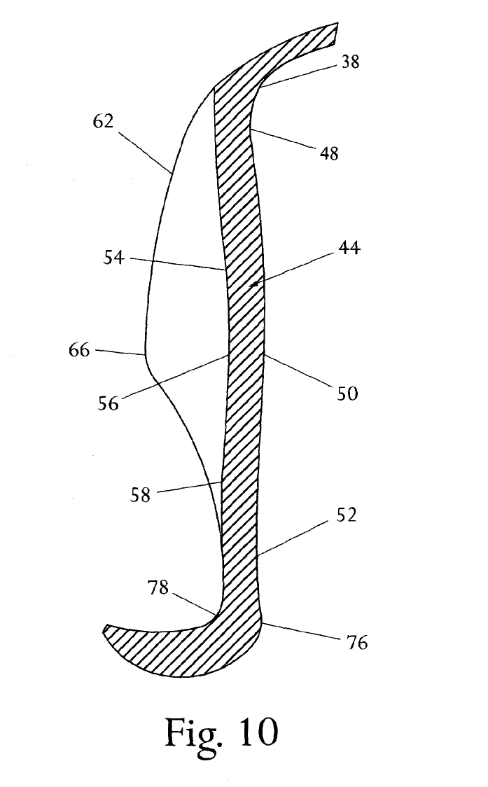

In an example embodiment, nasal implant 100 comprises body portion 44 operable to define central axis X (such as illustrated in FIGS. 5 and 7). Body portion 44 may comprise a relatively concave-shaped inner body surface (such as illustrated in FIGS. 2, 3, and 5) portion and a relatively convex-shaped outer body surface (such as illustrated in FIGS. 1, 4, and 6) portion. In example embodiments, a length between a lower portion and an upper portion of the body portion 44 may be between 36-44 mm, and a thickness between the inner body surface and the outer body surface of the body portion 44 may be between 0.1-5 mm. The inner body surface may comprise ventral groove portion 54 (see FIG. 10), which may be disposed substantially parallel to central axis X. The outer body surface may comprise ridge portion 46, which may also be disposed substantially parallel to central axis X. Ventral groove portion 54 may comprise one or more deep portions, and one or more of these deep portions may be in proximate correspondence with one or more high portions of ridge portion 46 with respect to central axis X. In an example embodiment, ventral groove portion 54 comprises deepest portion 56 and ridge portion 46 comprises highest portion 50, and said deepest portion 56 may be in proximate correspondence with highest portion 50 relative to central axis X. Ridge portion 46 may have upper portion 48 and lower portion 52. In example embodiments, a length between an upper portion and lower portion of ventral groove portion 54 may be between about 26-40 mm, and more preferably between about 30-35 mm, and a depth of deepest portion 56 of ventral groove portion 54 relative to a plane formable by a lower portion and upper portion of the inner body surface of body portion 44 may be between about 0.5-2 mm (see FIG. 10), and more preferably between about 0.7-1 mm. Furthermore, a length between upper portion 48 and lower portion 52 of ridge portion 46 may be between about 26-40 mm, and more preferably between about 30-35 mm, and a height of highest portion 50 of ridge portion 46 relative to a plane formable by a lower portion and upper portion of the outer body surface of body portion 44 may be between about 0.5-1 mm (see FIG. 10), and more preferably between about 0.7-1 mm. In operation, deepest portion 56 of ventral groove portion 54 is operable to receive at least a portion of a dorsal hump (not shown) of a recipient patients' nose when the nasal implant 100 is implanted.

Nasal implant 100 may also comprise tail portion 32 for contacting with at least a portion of a root of the nose (not shown) when nasal implant 100 is implanted. Tail portion 32 and body portion 44 may be formed together as a unitary article and/or fixedly attached to one another in example embodiments. A lower portion of tail portion 32 may be in communication with at least the upper portion of body portion 44. That is, a lower portion of an inner surface of tail portion 32 may form a substantially continuous surface with the lower portion of the inner body surface of body portion 44, and/or a lower portion of an outer surface of tail portion 32 may form a substantially continuous surface with the lower portion of the outer body surface of body portion 44. An angle α (see FIG. 7) formable between an axis definable by tail portion 32 and central axis X may be between about 0-90 degrees, and more preferably between about 15-75 degrees, and even more preferably between about 35-55 degrees. Tail portion 32 comprises lateral edges 40 and base corners 42, and may begin at upper portion 34 and slightly curve to form upper concave curve 36 and trough 38. In example embodiments, a length between upper portion 34 and the lower portion of tail portion 32 may be between about 3-12 mm, and more preferably between about 7-10 mm, and a thickness between the inner surface and the outer surface of tail portion 32 may be between about 0.1-2.5 mm. In operation, tail portion 32 may be adjustably operable to receive at least a portion of a glabella (not shown) of a recipient patient when nasal implant 100 is implanted. Tail portion 32 may be operable to fixably secure nasal implant 100 about at least a root (not shown) of the nose and assist in restricting and/or eliminating a movement of body portion 44 relative to at least the root of the nose when nasal implant 100 is implanted.

Example embodiments of the nasal implant 100 may further comprise one or more first wing portions 60. First wing portion 60 and body portion 44 may be formed together as a unitary article and/or fixedly attached to one another in example embodiments. First wing portion 60 may be formed as a unitary article to and/or fixedly attached with an outer portion of body portion 44 (such as the left side or the right side) at an inner portion of first wing portion 60. That is, an inner portion of an inner surface of first wing portion 60 may form a substantially continuous surface with an outer portion of the inner body surface of body portion 44, and/or an inner portion of an outer surface of first wing portion 60 may form a substantially continuous surface with an outer portion of the outer body surface of body portion 44.

As illustrated in the figures, nasal implant 100 may comprise two first wing portions 60, namely, a left first wing portion and a right first wing portion in communication with a left outer portion and a right outer portion, respectively, of body portion 44. In example embodiments, first wing portion 60 may comprise one or more inclines 62 along an outer portion (or protruding edge portion) of first wing portion 60, wherein the said outer portion of first wing portion 60 being opposite to the inner portion of first wing portion 60. The said outer portion of first wing portion 60 may be configurable to provide for a widest portion in or about one or more primary width points 66 and one or more width changing portions in or about point 64. In example embodiments, a length between the inner portion and the outer portion of first wing portion 60 may be between about 4-8 mm, and a thickness of the outer portion of each first wing portion 60 may be between about 0.1-1 mm. When nasal implant 100 comprises left first wing portion and right first wing portion, an angle 13 (see FIG. 8) formable between a plane definable by left first wing portion and a plane definable by right first wing portion may be between about 45-90 degrees, as illustrated in FIG. 8, and more preferably between about 55-80 degrees, and a distance between the outer portion of left first wing portion and the outer portion of right first wing portion, such as between the primary width points 66 of left first wing portion and right first wing portion, may be between about 12-17 mm. In operation, first wing portion 60 may be operable to adjustably receive at least a portion of a lateral cartilage (not shown) of a recipient patient when nasal implant 100 is implanted. First wing portion 60 and body portion 44 may be operable to cooperate together to restrict a movement of body portion 44 relative to at least central axis X when nasal implant 100 is implanted.

Nasal implant 100 may further comprise neck portion 70 in example embodiments. Neck portion 70 and body portion 44 may be formed together as a unitary article and/or fixedly attached to one another in example embodiments. Neck portion 70 may be formed as a unitary article to and/or fixedly attached with the lower portion of body portion 44 at an upper portion of neck portion 70. That is, an upper portion of an inner surface of neck portion 70 may form a substantially continuous surface with the lower portion of the inner body surface of body portion 44, and/or an upper portion of an outer surface of neck portion 70 may form a substantially continuous surface with the lower portion of the outer body surface of body portion 44. Neck portion 70 may further comprise narrow portion 72. In example embodiments, a width of neck portion 70 at narrow portion 72 may be between about 0.6-4 mm, and more preferably between about 0.6-3.25 mm, a length of neck portion 70 between the upper portion of neck portion 70 and a lower portion of neck portion 70 may be between about 6-10 mm, and more preferably between 7-9 mm, and a thickness between the inner surface and the outer surface of neck portion 70 may be between about 0.1-4 mm. In operation, neck portion 70 may be operable to secure body portion 44 with head portion 74, while enabling natural movement of head portion 74 relative to body portion 44.

In example embodiments, nasal implant 100 may further comprise head portion 74. Head portion 74, neck portion 70, and body portion 44 may be formed together as a unitary article and/or fixedly attached to one another in example embodiments. Head portion 74 may be formed as a unitary article to and/or fixedly attached with the lower portion of neck portion 70 at an upper portion of head portion 74. Head portion 74 comprises an inner head surface and an outer head surface. The inner head surface of head portion 74 may be operable to form a substantially continuous surface with a lower portion of the inner surface of neck portion 70 and/or the lower portion of the inner body surface of body portion 44. Similarly, the outer head surface of head portion 74 may be operable to form a substantially continuous surface with a lower portion of the outer surface of neck portion 70 and/or the lower portion of the outer body surface of body portion 44. In example embodiments, head portion 74 may comprise outer protrusion portion 76 and inner cavity portion 78. In example embodiments, a width between a left outer portion and a right outer portion of head portion 74 may be between about 12-17 mm, and a thickness between the inner head surface and the outer head surface may be between about 0.1-5 mm. Furthermore, an angle θ (see FIG. 7) formable between an axis definable by head portion 74 and central axis X may be greater than about 15 degrees, as illustrated in FIG. 7. In operation, a deepest portion of inner cavity portion 78 may be operable to receive at least a portion of a greater alar cartilage (not shown) when nasal implant 100 is implanted.

Nasal implant 100 may further comprise one or more second wing portions 80. Second wing portion 80 and head portion 74 may be formed together as a unitary article and/or fixedly attached to one another in example embodiments. Second wing portion 80 may be formed as a unitary article to and/or fixedly attached with an outer portion of the head portion 74 at an inner portion of second wing portion 80. As illustrated in the figures, nasal implant 100 may comprise two second wing portions 80, namely, a left second wing portion and a right second wing portion in communication with a left outer portion and a right outer portion, respectively, of head portion 74. Second wing portion 80 may further comprise an inner second wing surface operable to form a substantially continuous surface with the inner head surface of head portion 74. Correspondingly, second wing portion 60 may comprise an outer second wing surface operable to form a substantially continuous surface with the outer head surface of head portion 74. In example embodiments, a length between the inner portion and an outer portion (outer peripheral edge) of each second wing portion 80 may be between about 2-4 mm, and a thickness of the outer portion of each second wing portion 80 may be between about 0.1-1 mm. When nasal implant 100 comprises left second wing portion and right second wing portion, a distance between the outer portion of left second wing portion and the outer portion of right second wing portion may be between about 4-8 mm. In operation, second wing portion 80 may be adjustably operable to receive at least a portion of an alar cartilage (not shown) of a recipient patient when nasal implant 100 is implanted. Second wing portion 80 and head portion 74 may be operable to cooperate together to restrict a movement of body portion 44 when nasal implant 100 is implanted.

Furthermore, nasal implant 100 may comprise leg portion 84. Leg portion 84 and head portion 74 may be formed together as a unitary article and/or fixedly attached to one another in example embodiments. Leg portion 84 may be formed as a unitary article to or fixedly attached with a lower portion of head portion 74 at an upper portion of leg portion 84. Leg portion 84 may also comprise an inner leg surface operable to form a substantially continuous surface with the inner head surface of head portion 74, and/or an outer leg surface operable to form a substantially continuous surface with the outer head surface of head portion 74. In example embodiments, a length between the upper portion and a lower portion of leg portion 84 may be between about 3-7 mm, and a thickness between the inner surface and the outer surface of leg portion 84 may be between about 0.1-2.5 mm. Preferably, a width of the upper portion of leg portion 84 is greater than a width of the lower portion of leg portion 84. In operation, the inner surface of leg portion 84 may be operable to receive at least a portion of a columella (not shown) of a recipient patient when nasal implant 100 is implanted. Leg portion 84 may be operable to cooperate together with head portion 74 to restrict a movement of head portion 74 when nasal implant 100 is implanted.

Nasal implant 100 may be configurable in one of a plurality of ways. Configuration of nasal implant 100 may be based on one or more factors, including patients' condition, need, desires, and/or structure/shape/size of the patients' nasal areas. In an example embodiment, nasal implant 100 may comprise body portion 44 and tail portion 32. In another example embodiment, nasal implant 100 may comprise body portion 44 and one or more first wing portions 60. In another example embodiment, nasal implant 100 may comprise body portion 44, tail portion 32, and one or more first wing portions 60. In another example embodiment, nasal implant 100 may comprise body portion 44 and head portion 74. The said embodiment may further comprise neck portion 70 between body portion 44 and head portion 74. In another example embodiment, nasal implant 100 may comprise body portion 44, head portion 74, and one or more second wing portions 80. The said embodiment may further comprise neck portion 70 between body portion 44 and head portion 74. In another example embodiment, nasal implant 100 may comprise body portion 44, head portion 74, and leg portion 84. The said embodiment may further comprise neck portion 70 between body portion 44 and head portion 74. In another example embodiment, nasal implant 100 may comprise body portion 44, head portion 74, one or more second wing portions 80, and leg portion 84. The said embodiment may further comprise neck portion 70 between body portion 44 and head portion 74. In another example embodiment, nasal implant 100 may comprise body portion 44, tail portion 32, head portion 74, one or more first wing portions 60, one or more second wing portions 80, leg portion 84, and neck portion 70.

One or more of body portion 44, tail portion 32, head portion 74, first wing portion 60, second wing portion 80, leg portion, and neck portion 70 may be formed of a material composition that is safe and/or approved for medical implantation use, such as silicon. Known types of silicon for use with example embodiments of nasal implant 100 may include polytetrafluoroethylene (ePTFE), polyethylene, and hydroxyapatite.

In another example embodiment, a method is described for adapting nasal implant 100 for nasal augmentation. Nasal implant 100 comprises elongated body portion 44, elongated tail portion 32 in communication with at least an upper portion of body portion 44, first wing portion 60 in communication with at least a first outer portion of body portion 44, head portion 74 in communication with at least a lower portion of body portion 44, second wing portion 80 in communication with at least an outer portion of head portion 74, and leg portion 84 in communication with at least a lower portion of head portion 74. Body portion 44 forms central axis X and comprises opposing inner body and outer body surfaces, the inner body surface having ventral groove portion 54 disposed substantially parallel to central axis X, the outer body surface having ridge portion 46, and ventral groove portion 54 comprising deepest portion 56 in proximate correspondence to highest portion 50 of ridge portion 46 with respect to central axis X. The method comprises identifying an appropriate set of dimensions of nasal implant 100 for the nasal augmentation, and adapting nasal implant 100 for the nasal augmentation by performing adjusting and/or configuring, including trimming, of at least an outer peripheral portion of at least one of body portion 44, tail portion 32, first wing portion 60, neck portion 70, second wing portion 80, head portion 74, and leg portion 84.

In another example embodiment, a method is described for adapting nasal implant 100 for nasal augmentation. Nasal implant 100 comprises elongated body portion 44, elongated tail portion 32 in communication with at least an upper portion of body portion 44, first wing portion 60 in communication with at least a first outer portion of body portion 44, head portion 74 in communication with at least a lower portion of body portion 44, second wing portion 80 in communication with at least an outer portion of head portion 74, and leg portion 84 in communication with at least a lower portion of head portion 74. Body portion 44 forms central axis X and comprises opposing inner body and outer body surfaces, the inner body surface having ventral groove portion 54 disposed substantially parallel to central axis X, the outer body surface having ridge portion 46, and ventral groove portion 54 comprising deepest portion 56 in proximate correspondence to highest portion 50 of ridge portion 46 with respect to central axis X. The method comprises identifying an appropriate set of dimensions of nasal implant 100 for the nasal augmentation, and adapting nasal implant 100 for the nasal augmentation by detaching at least one of body portion 44, tail portion 32, first wing portion 60, neck portion 70, second wing portion 80, head portion 74, and leg portion 84.

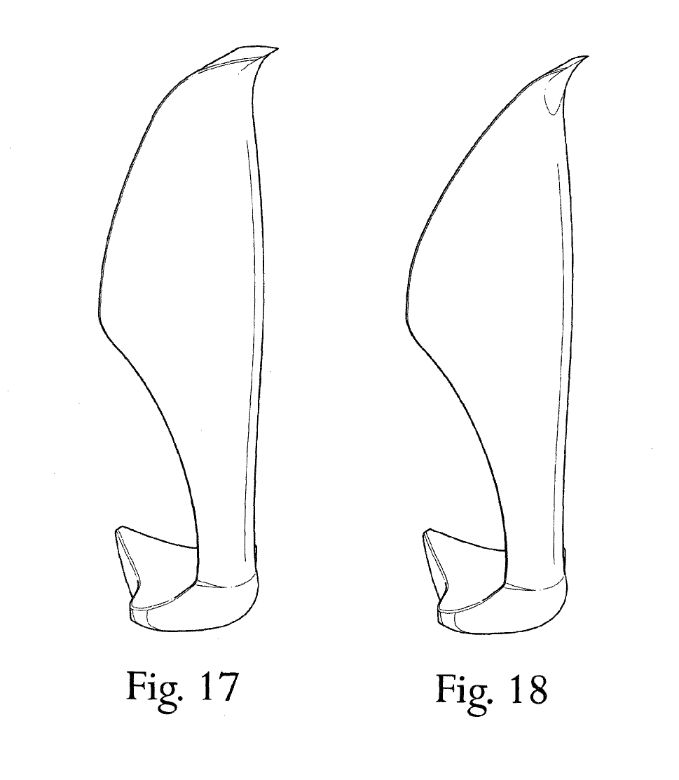

Tail portion 32 may be trimmed or detached entirely from nasal implant 100. In patients requiring less augmentation via tail portion 32, tail portion 32 may be adjusted and/or configured, such as by trimming, to be shorter in length and thinner in or about the outer portions. FIGS. 17 and 18 depict two possible results of a tail portion 32 after trimming. Angle α may be increased through adjusting, such as slicing, of the inner surface of tail portion 32.

Ridge portion 46 of body portion 44 may be adjusted and/or configured, such as by trimming, based on the dorsal hump (not shown) of the recipient patient. For example, if the recipient patient does not require much or any augmentation due to a sufficiently shaped or curved dorsal hump, then ridge portion 46 may be trimmed accordingly.

Ventral groove 54 of body portion may be adjusted and/or configured, such as by trimming, based on the dorsal hump of the recipient as well.

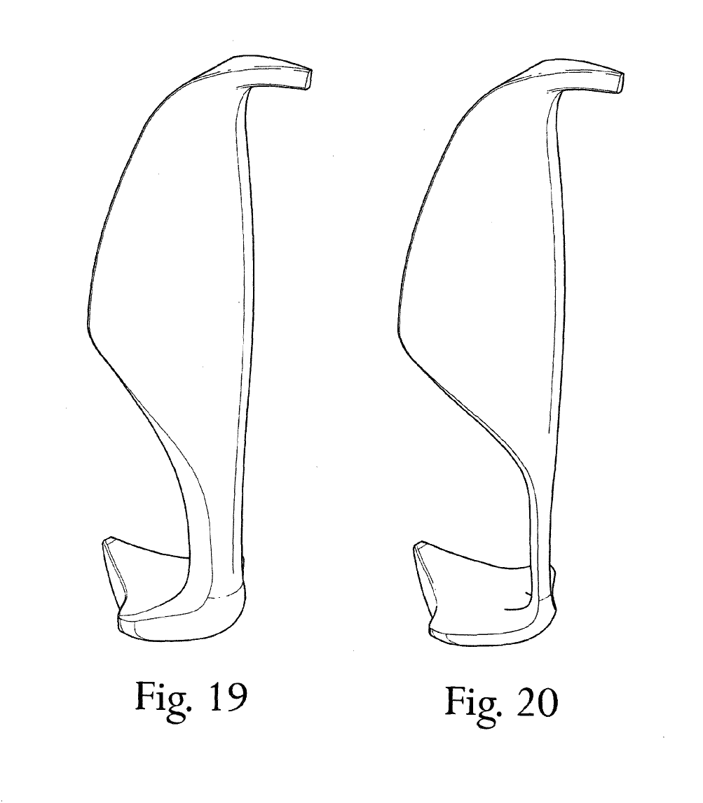

In patients requiring little or no augmentation in the area of the nose corresponding to neck portion 70 of nasal implant 100, neck portion 70 may be adjusted and/or configured, such as by trimming, to be very thin. FIGS. 19 and 20 depict two possible, resultant shapes of neck portion 70 after trimming. Trimming down of neck portion 70 may provide a more naturally flexible nose after nasal implant 100 is implanted.

In patients requiring little or no augmentation in the area of the nose corresponding to the tip of the nose, head portion 74 may be adjusted and/or configured, such as by trimming FIG. 20 provides an example of trimming head portion 74 to be very thin. Head portion 74 may be adjusted, such as by slicing, from the upper portion to effectively gain more operative height of nasal implant 100, or adjusted, such as by slicing, from its underside to retain the overall length of nasal implant 100.

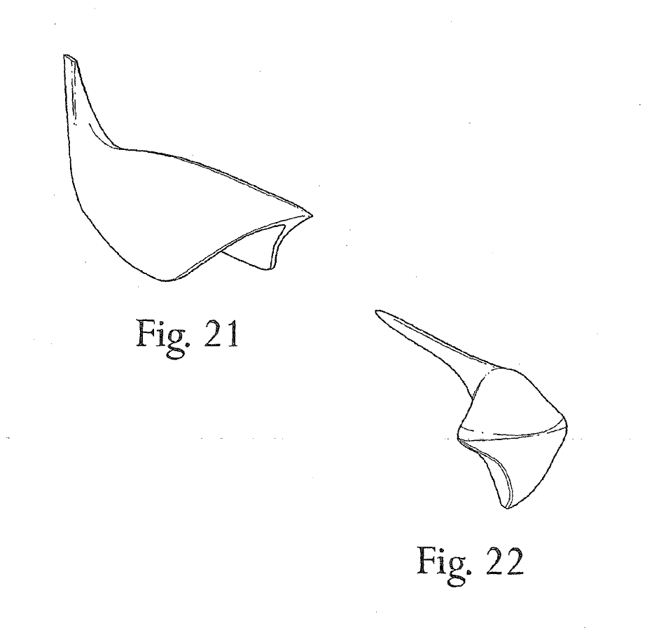

In certain patients wherein the dorsal length of the nose may be long, such as in situation wherein the dorsal length is longer than the length of nasal implant 100, adjustment around narrow point 72, end point 58, and/or lower point 52 may be performed, such as by slicing in part or entirely, in order to gain more length and dichotomize nasal implant 100 into two parts. FIGS. 21 and 22 depict one possible result of this dichotomy. In these cases, the one or more portions may be adjusted into appropriate shapes that can be easily held by the recipient patients' tissues after healing. The surgeon should also trim any parts that are not neat, such as thick ends and sharp corners. The shapes for holding can be crafted elsewhere—an upper end of neck portion 70, which adjoins head portion 74, for example.

Some additional embodiments may be achieved by modifications of one or more features of nasal implant 100. An additional embodiment is illustrated here merely as an example of numerous additional embodiments for enhancement of the reader's understanding. Said additional embodiment is characterized in that the angle 13 between greater wings may be about 110 degree, and the three distances between opposite subordinate width points 64, primary width points 66, and secondary width points 82 may be about 17 mm.

There are multitudinous possibilities in regard to variations on quantity, shape, location, dimension, and substance. Several embodiments and their parts, however small, comprise variations on one or more aspects delineated as follows. The quantity of each portion of the nasal implant 100 may vary. For example, two or more tail portions 32, body portions 44, ridge portions 46, ventral groove portions 54, first wing portions 60, neck portions 70, head portions 74, second wing portions 80, leg portions 84, or any other portions, however small, may be incorporated into the nasal implant 100 as described above and herein in example embodiments. Alternatively or in addition, a reduction in the quantity of each portion may also be incorporated into nasal implant 100 as described above and herein in example embodiments. For example, one or no tail portion 32, body portion 44, ridge portion 46, ventral groove portion 54, first wing portion 60, neck portion 70, head portion 74, second wing portion 80, leg portion 84, or any other portion, however small, may be incorporated into nasal implant 100 as described above and herein in example embodiments.

In other example embodiments, one or more parts, however small, may be positioned in one or more other locations in or about nasal implant 100. For example, primary width points 66 may be positioned at other locations on first wing portions 60; ventral groove portion 54 and/or ridge portion 46 may be provided elsewhere on the respective surfaces of body portion 44 to provide better fit on the recipient patient; second wing portion 80 may attach to head portion 74 at other portions; highest point 50, deepest point 56, and primary width points 66 may be in different locations as well.

In other example embodiments, variations to the shape of nasal implant 100 may be provided to one or more portions. For example, tail portion 32 may be one or more of a rectangular shape, pentagonal shape, hexagonal shape, circular shape, and/or any other geometrical shapes, asymmetrical shapes, inclining shapes, and the like. Body portion 44 may be one or more of a straight body, convex body, concave body, protruding body, receding body, body without ventral groove portion 54, body without ridge portion 46, or without both, stepped body, inclining body, any other geometric bodies, and bodies of any other shapes. Ridge portion 46 may be one or more shapes, including triangular, rectangular, pentagonal, hexagonal, and the like. Ventral groove portion 54 may be one or more shapes, including triangular, rectangular, pentagonal, hexagonal, or any other geometric shapes. First wing portion 60 may be without lower concave curves and may be of any other shapes. Neck portion 70 may be a uniform neck, hourglass-shaped neck, stepped neck, inclining neck, and neck of any other shapes. Head portion 74 may be one or more of a beveled head and any other shape. Second wing portion 80 may be one or more of a rectangular shape, pentagonal shape, hexagonal shape, circular shape, and any other geometric shapes. Leg portion 84 may be one or more of a straight leg, triangular leg, rectangular leg, pentagonal leg, hexagonal leg, any other geometric legs, leg with base or bases at its end, and legs of any other shapes are possible. Furthermore, shapes on any portions may also be possible—for example, making one or more holes, cavities, protrusions, and/or channels, however small, cutting, hollowing, and making patterns or shapes for better holding.

In example embodiments, deviations, regardless of extent, from one or more dimensions specified of each portion, however small, including height, width, depth, length, thickness, and angle may be applied to nasal implant 100.

In example embodiments, the portions of nasal implant 100 may be of heterogeneous and/or homogenous composition. For example, an example embodiment of nasal implant 100 may comprise head portion 74 having soft-grade silicone composition, whereas the rest of nasal implant 100 may comprise hard-grade silicone. As another example, nasal implant 100 may be impregnated with other substances. Any substances that do not induce malign effects to the nasal tissues can be elected—regardless of their physical properties, such as hardness, roughness, flexibility, and being porous. The four substances described above and herein, namely silicone, expanded polytetrafluoroethylene (ePTFE), polyethylene, and hydroxyapatite, are merely examples of various substances that may be for use in nasal implant 100.

It is recognized herein that present example embodiments provide for one or more of the following advantages: preservation of one or more sizes of angles and continuous curves at the roots of noses; no conspicuous depressions; resultant nasal dorsa will be relatively narrow and straight; nasal implants will be less susceptible to displacement, rotation, and inclining; resultant noses will be more naturally flexible, providing natural looks for the patient; less probable to perforation at the tips of noses and to sink and break into greater alar cartilages; less excessive weight, and, hence, patients can feel more comfortable; nasal implants are easily and conveniently adaptable and customizable so as to cope with the wide variation on shapes of noses; and enables recipient patients' own surrounding nasal tissues to recover (heal) and grow around the nasal implant so as to provide support of the nasal implant.

While various embodiments in accordance with the disclosed principles have been described above, it should be understood that they have been presented by way of example only, and are not limiting. Thus, the breadth and scope of the example embodiments described herein should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

Various terms used herein have special meanings within the present technical field. Whether a particular term should be construed as such a “term of art” depends on the context in which that term is used. “Connected to,” “in communication with,” “associated with,” or other similar terms should generally be construed broadly to include situations both where connections and/or attachments are direct between referenced elements or through one or more intermediaries between the referenced elements. Furthermore, “deep portion”, “depressions”, “recessed portion” or other similar terms should generally be construed broadly to include a cavity, a trough, a bore, a recess, a hole, and/or any other indentation, or the like, formed by a surface. Furthermore, “protruded member,” “elongated member,” or other similar terms should generally be construed broadly to include an elongated body, a protrusion, a peak, and/or any other elongated or protruded surface or body having smooth and/or angled surfaces. These and other terms are to be construed in light of the context in which they are used in the present disclosure and as one of ordinary skill in the art would understand those terms in the disclosed context. The above definitions are not exclusive of other meanings that might be imparted to those terms based on the disclosed context.

Words of comparison, measurement, and timing such as “at the time,” “equivalent,” “during,” “complete,” and the like should be understood to mean “substantially at the time,” “substantially equivalent,” “substantially during,” “substantially complete,” etc., where “substantially” means that such comparisons, measurements, and timings are practicable to accomplish the implicitly or expressly stated desired result.

Additionally, the section headings in the present disclosure are provided for consistency with the suggestions under 37 C.F.R. 1.77 or otherwise to provide organizational cues. These headings shall not limit or characterize the invention(s) set out in any claims that may issue from this disclosure. Specifically, a description of a technology in the “Background” section shall not be construed as an admission that technology is prior art to any invention(s) in this disclosure. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings in the present disclosure.

1. A nasal implant, comprising:
an elongated body portion, the body portion forming a central axis and having opposing inner body and outer body surfaces, the inner body surface having a ventral groove portion disposed substantially parallel to the central axis, and the outer body surface having a ridge portion, wherein the ventral groove portion comprises a deepest portion in proximate correspondence to a highest portion of the ridge portion with respect to the central axis;
an elongated tail portion, a lower portion of the tail portion in communication with at least an upper portion of the body portion, wherein an angle formable between an axis definable by the tail portion and the central axis is between 0-90 degrees;
a first wing portion, the first wing portion having opposing inner first wing and outer first wing surfaces, wherein an inner portion of the first wing portion is in communication with at least a first outer portion of the body portion;
a head portion, the head portion having opposing inner head and outer head surfaces, wherein the inner head surface is in communication with at least a lower portion of the inner body surface and the outer head surface is in communication with at least a lower portion of the outer body surface;
a second wing portion, the second wing portion having opposing inner second wing and outer second wing surfaces, wherein an inner portion of the second wing portion is in communication with at least a first outer portion of the head portion; and
a leg portion, the leg portion having opposing inner leg and outer leg surfaces, an upper portion of the leg portion in communication with at least a lower portion of the head portion.

2. The nasal implant of claim 1, wherein the deepest portion of the ventral groove portion is operable to receive at least a portion of a dorsal hump when the nasal implant is implanted.

3. The nasal implant of claim 1, wherein a length between a lower portion of the body portion and the upper portion of the body portion is between about 36-44 mm.

4. The nasal implant of claim 1, wherein a thickness between the inner body surface and the outer body surface is between about 0.1-5 mm.

5. The nasal implant of claim 1, wherein the ventral groove portion extends substantially between a lower portion of the body portion and the upper portion of the body portion.

6. The nasal implant of claim 1, wherein a length of the ventral groove portion is between about 26-40 mm.

7. The nasal implant of claim 1, wherein a depth of the deepest portion of the ventral groove portion relative to a plane formable by the lower portion of the inner body surface and an upper portion of the inner body surface is between about 0.5-2 mm.

8. The nasal implant of claim 1, wherein a height of the highest portion of the ridge portion relative to a plane formable by the lower portion of the outer body surface and an upper portion of the outer body surface is between about 0.5-1 mm.

9. The nasal implant of claim 1, wherein the tail portion is adjustably operable to receive at least a portion of a glabella when the nasal implant is implanted.

10. The nasal implant of claim 1, wherein the tail portion is operable to fixably secure the nasal implant at a root of the nose and restrict a movement of the body portion relative to the root when the nasal implant is implanted.

11. The nasal implant of claim 1, wherein a width of an upper portion of the tail portion is less than a width of the lower portion of the tail portion.

12. The nasal implant of claim 1, wherein a length between an upper portion of the tail portion and the lower portion of the tail portion is between about 3-12 mm.

13. The nasal implant of claim 1, wherein a thickness of the tail portion is between about 0.1-2.5 mm.

14. The nasal implant of claim 1, wherein the first wing portion is adjustably operable to receive at least a portion of a lateral cartilage when the nasal implant is implanted.

15. The nasal implant of claim 1, wherein the first wing portion and the body portion cooperate to restrict a movement of the body portion relative to the central axis when the nasal implant is implanted.

16. The nasal implant of claim 1, wherein an outer peripheral portion of the first wing portion opposite the inner portion of the first wing portion comprises a protruding edge portion, wherein a length between the inner portion of the first wing portion and the protruding edge portion is between about 4-8 mm.

17. The nasal implant of claim 16, wherein a thickness of the outer peripheral portion of the first wing portion opposite the inner portion of the first wing portion is between about 0.1-1 mm.

18. The nasal implant of claim 1, further comprising a second first wing portion, the second first wing portion comprising an inner portion of the second first wing portion in communication with at least a second outer portion of the body portion opposite the first outer portion of the body portion.

19. The nasal implant of claim 18, wherein an angle formable between a plane definable by the first wing portion and a plane definable by the second first wing portion is between about 45-110 degrees.

20. The nasal implant of claim 18, wherein the second first wing portion is adjustably operable to receive at least a portion of a lateral cartilage when the nasal implant is implanted.

21. The nasal implant of claim 18, wherein the second first wing portion, the first wing portion, and the body portion cooperate to restrict a movement of the body portion relative to the central axis when the nasal implant is implanted.

22. The nasal implant of claim 18, wherein an outer peripheral portion of the second first wing portion opposite the inner portion of the second first wing portion comprises a protruding edge portion, wherein a length between the inner portion of the second first wing portion and the protruding edge portion of the second first wing portion is between about 4-8 mm.

23. The nasal implant of claim 22, wherein a thickness of the outer peripheral portion of the second first wing portion opposite the inner portion of the second first wing portion is between about 0.1-1 mm.

24. The nasal implant of claim 22, wherein a distance between the protruding edge of the second first wing portion and a corresponding protruding edge of the first wing portion is between about 12-17 mm.

25. The nasal implant of claim 1, further comprising a neck portion, wherein an upper portion of the neck portion is in communication with at least a lower portion of the body portion and a lower portion of the neck portion is in communication with at least an upper portion of the head portion.

26. The nasal implant of claim 25, wherein a width of the neck portion is between about 0.6-4 mm.

27. The nasal implant of claim 25, wherein a thickness of the neck portion is between about 0.1-4 mm.

28. The nasal implant of claim 1, wherein a deepest portion of the inner head surface is operable to receive at least a portion of a greater alar cartilage when the nasal implant is implanted.

29. The nasal implant of claim 1, wherein a width between a first and second outer portion of the head portion is between about 12-17 mm.

30. The nasal implant of claim 1, wherein a thickness between the inner head surface and the outer head surface is between about 0.1-5 mm.

31. The nasal implant of claim 1, wherein an angle formable between an axis definable by the head portion and the central axis is greater than about 15 degrees.

32. The nasal implant of claim 1, wherein the outer head surface comprises a protruded portion.

33. The nasal implant of claim 1, wherein an inner leg surface of the leg portion is operable to receive at least a portion of a columella when the nasal implant is implanted.

34. The nasal implant of claim 1, wherein the leg portion is operable to cooperate with the head portion to restrict a movement of the head portion when the nasal implant is implanted.

35. The nasal implant of claim 1, wherein a length of the leg portion is between about 3-7 mm.

36. The nasal implant of claim 1, wherein a width of the upper portion of the leg portion is greater than a width of a lower portion of the leg portion.

37. The nasal implant of claim 1, wherein a thickness of the leg portion is between about 0.1-2.5 mm.

38. The nasal implant of claim 1, wherein the second wing portion is adjustably operable to receive at least a portion of an alar cartilage when the nasal implant is implanted.

39. The nasal implant of claim 1, wherein the second wing portion and the head portion cooperate to restrict a movement of the body portion when the nasal implant is implanted.

40. The nasal implant of claim 1, wherein an outer peripheral portion of the second wing portion opposite the inner portion of the second wing portion comprises a protruding edge portion, wherein a length between the inner portion of the second wing portion and the protruding edge portion is between about 2-4 mm.

41. The nasal implant of claim 40, wherein a thickness of the outer peripheral portion of the second wing portion opposite the inner portion of the second wing portion is between about 0.1-1 mm.

42. The nasal implant of claim 1, further comprising a second second wing portion, the second second wing portion comprising an inner portion of the second second wing portion in communication with at least a second outer portion of the head portion opposite the first side portion of the head portion.

43. The nasal implant of claim 42, wherein the second second wing portion is adjustably operable to receive at least a portion of an alar cartilage when the nasal implant is implanted.

44. The nasal implant of claim 42, wherein the second second wing portion, the second wing portion, and the head portion cooperate to restrict a movement of the head portion when the nasal implant is implanted.

45. The nasal implant of claim 42, wherein an outer peripheral portion of the second second wing portion opposite to the inner portion of the second second wing portion comprises a protruding edge portion, wherein a length between the inner portion of the second second wing portion and the protruding edge portion of the second second wing portion is between about 2-4 mm.

46. The nasal implant of claim 42, wherein a thickness of the outer peripheral portion of the second second wing portion opposite the inner portion of the second second wing portion is between about 0.1-1 mm.

47. The nasal implant of claim 1, wherein the body portion and the first wing portion are integrally formed as a unitary article.

48. The nasal implant of claim 1, wherein the body portion and the tail portion are integrally formed as a unitary article.

49. The nasal implant of claim 1, wherein the body portion and the head portion are integrally formed as a unitary article.

50. The nasal implant of claim 1, wherein the body portion is in communication with the head portion via a neck portion, the neck portion being integrally formed with the body portion and the head portion as a unitary article.

51. The nasal implant of claim 1, wherein the head portion and the second wing portion are integrally formed as a unitary article.

52. The nasal implant of claim 1, wherein the head portion and the leg portion are integrally formed as a unitary article.

53-120. (canceled)

121. A method of adapting a nasal implant for nasal augmentation, the nasal implant having an elongated body portion, an elongated tail portion in communication with at least an upper portion of the body portion, a first wing portion in communication with at least a first outer portion of the body portion, a head portion in communication with at least a lower portion of the body portion, a second wing portion in communication with at least an outer portion of the head portion, and a leg portion in communication with at least a lower portion of the head portion, the body portion forming a central axis and having opposing inner body and outer body surfaces, the inner body surface having a ventral groove portion disposed substantially parallel to the central axis, the outer body surface having a ridge portion, and the ventral groove portion comprising a deepest portion in proximate correspondence to a highest portion of the ridge portion with respect to the central axis, the method comprising:
identifying an appropriate set of dimensions of the nasal implant for the nasal augmentation; and
adapting the nasal implant for the nasal augmentation by performing a trimming of at least an outer peripheral portion of at least one of:
the body portion;
the tail portion;
the first wing portion;
the neck portion;
the second wing portion;
the head portion; and
the leg portion.

122. The method of claim 121, wherein the deepest portion of the ventral groove is operable to receive at least a portion of a dorsal hump when at least the outer peripheral portion of the body portion is trimmed and the nasal implant is implanted.

123. The method of claim 121, wherein the tail portion is operable to fixably secure the nasal implant at a root of the nose and restrict a movement of the body portion relative to the root when at least the outer peripheral portion of the tail portion is trimmed and the nasal implant is implanted.

124. The method of claim 121, wherein the tail portion is operable to receive at least a portion of a glabella when at least the outer peripheral portion of the tail portion is trimmed and the nasal implant is implanted.

125. The method of claim 121, wherein the first wing portion is operable to receive at least a portion of a lateral cartilage when at least the first wing portion is trimmed and the nasal implant is implanted.

126. The method of claim 121, wherein the first wing portion and the body portion cooperate to restrict a movement of the body portion relative to the central axis when the nasal implant is implanted.

127. The method of claim 121, further comprising trimming at least an outer peripheral portion of a second first wing portion.

128. The method of claim 121, wherein an inner surface of the head portion is operable to receive at least a portion of a greater alar cartridge when at least the head portion is trimmed and the nasal implant is implanted.

129. The method of claim 121, wherein an inner surface of the leg portion is operable to receive at least a portion of a columella when the leg portion is trimmed and the nasal implant is implanted.

130. The method of claim 121, wherein the second wing portion is operable to receive at least a portion of an alar cartilage when the second wing portion is trimmed and the nasal implant is implanted.

131. The method of claim 121, wherein the second wing portion and the head portion cooperate to restrict a movement of the body portion when the nasal implant is implanted.

132. The method of claim 121, further comprising trimming at least an outer peripheral portion of a second second wing portion.

133. The method of claim 121, wherein the outer peripheral portion comprises at least a portion of a side edge.

134. The method of claim 121, wherein the outer peripheral portion comprises at least a portion of a surface.

135. The method of claim 121, wherein the trimming comprises reducing a thickness of a portion of the nasal implant.

136. The method of claim 121, wherein the trimming comprises reducing one or more dimensions of one or more of the body portion, the tail portion, the first wing portion, the tail portion, the head portion, the second wing portion, and the leg portion to correspond with one or more of the identified dimensions.

137. The method of claim 121, wherein the trimming comprises cutting.

138. The method of claim 121, wherein the adapted nasal implant provides for the nasal augmentation when the adapted nasal implant is implanted.

139. The method of claim 121, wherein the identifying is based on identifying at least a location of one or more of a dorsal hump, a root of the nose, a glabella, a lateral cartilage, a greater alar cartridge, a columella, and an alar cartilage.

140. The method of claim 121, wherein the identifying is based on identifying at least a size of one or more of a dorsal hump, a root of the nose, a glabella, a lateral cartilage, a greater alar cartridge, a columella, and an alar cartilage.

141. A method of adapting a nasal implant for nasal augmentation, the nasal implant having an elongated body portion, an elongated tail portion in communication with at least an upper portion of the body portion, a first wing portion in communication with at least a first outer portion of the body portion, a head portion in communication with at least a lower portion of the body portion, a second wing portion in communication with at least an outer portion of the head portion, and a leg portion in communication with at least a lower portion of the head portion, the body portion forming a central axis and having opposing inner body and outer body surfaces, the inner body surface having a ventral groove portion disposed substantially parallel to the central axis, the outer body surface having a ridge portion, and the ventral groove portion comprising a deepest portion in proximate correspondence to a highest portion of the ridge portion with respect to the central axis, the method comprising:
identifying an appropriate set of dimensions of the nasal implant for the nasal augmentation; and
adapting the nasal implant for the nasal augmentation by detaching at least one of:
the body portion;
the tail portion;
the first wing portion;
the neck portion;
the second wing portion;
the head portion; and
the leg portion.

142. The method of claim 141, wherein the detaching comprises cutting.

143. The method of claim 141, wherein the adapted nasal implant provides for the nasal augmentation when the adapted nasal implant is implanted.

144. The method of claim 141, wherein the adapted nasal implant and the at least one detached portion provide for nasal augmentation when the adapted nasal implant and the detached at least one portion are implanted.

145. The method of claim 141, wherein the identifying is based on identifying at least a location of one or more of a dorsal hump, a root of the nose, a glabella, a lateral cartilage, a greater alar cartridge, a columella, and an alar cartilage.

146. The method of claim 141, wherein the identifying is based on identifying at least a size of one or more of a dorsal hump, a root of the nose, a glabella, a lateral cartilage, a greater alar cartridge, a columella, and an alar cartilage.
